(19) United States
(12) Patent Application Publication
(10) Pub. No.: US 2024/0303879 A1
(43) Pub. Date: Sep. 12, 2024

(54) MAGNETIC PARTICLE IMAGING (MPI) RECONSTRUCTION METHOD BASED ON TIME-DOMAIN SYSTEM MATRIX AND X-SPACE

(71) Applicant: Shandong University, Jinan City (CN)

(72) Inventors: Xiaopeng MA, Jinan City (CN); Shihao SHAN, Jinan City (CN); Dexin YU, Jinan City (CN); Xiaoli YANG, Jinan City (CN); Min LI, Jinan City (CN)

(21) Appl. No.: 18/434,529

(22) Filed: Feb. 6, 2024

(30) Foreign Application Priority Data
Mar. 6, 2023 (CN) 202310200590.1

(51) Int. Cl. G06T 11/00; G01R 33/12

(52) U.S. Cl. CPC G06T 11/006; G01R 33/1276

(57) ABSTRACT

Provided is a magnetic particle imaging (MPI) reconstruction method based on a time-domain system matrix and x-space, including: obtaining a voltage signal through MPI scanning based on a Cartesian trajectory; obtaining an original image according to an x-space-based reconstruction method; constructing, based on the voltage signal as well as a velocity compensation step and a grading step in the x-space-based reconstruction method, a forward model for time-domain-based MPI and x-space-based reconstruction; taking the original image as an input of an inverse problem solver of the forward model, and obtaining an optimized particle distribution diagram through solving. Based on the original image obtained through the x-space-based reconstruction, the forward model describing MPI and x-space-based reconstruction processes is established. An iterative reconstruction algorithm is used to eliminate an impact of a point spread function on an image obtained through the x-space-based reconstruction, achieving MPI image reconstruction with isotropic resolution and no artifact.

### CROSS REFERENCE TO RELATED APPLICATION

This patent application claims the benefit of and priority to Chinese Patent Application No. 202310200590.1, filed with the Chinese Patent Office on Mar. 6, 2023, which is hereby incorporated by reference herein in its entirety.

### TECHNICAL FIELD

The present disclosure relates to the technical field of magnetic particle imaging (MPI), and specifically to an MPI reconstruction method based on a time-domain system matrix and x-space.

### BACKGROUND

As a rising molecular imaging technology, the magnetic particle imaging (MPI) technology can noninvasively detect a distribution of superparamagnetic magnetic nanoparticles in a living body, and has advantages such as high sensitivity, no background interference, fast dynamic imaging, no impact from a depth of a tissue being detected, and no ionizing radiation.

MPI reconstruction is to recover a voltage signal from a receiving coil of an MPI device to a particle concentration image. There are currently two mainstream MPI reconstruction methods: a system matrix-based reconstruction method and an x-space-based reconstruction method. The x-space-based reconstruction method performs velocity normalization on the received voltage signal, meshes the voltage signal, maps a meshed voltage signal onto a scanning trajectory of a field free point (FFP), and models a reconstructed image as a convolution result of an original particle concentration distribution and a point spread function (PSF). In order to eliminate an impact of the PSF on the reconstructed image, two methods are provided to optimize an image obtained through x-space-based reconstruction: a deconvolution method and a multi-channel signal collection method. In the deconvolution method, for the image obtained through the x-space-based reconstruction, a deconvolution operation is performed based on a convolution kernel of the PSF by using a Wiener deconvolution method to eliminate the impact of the PSF. In the multi-channel signal collection method, for two-dimensional imaging, excitation and signal collection are separately performed in two orthogonal directions, and the x-space-based reconstruction is performed on voltage signals obtained through the excitation in the two directions. Then, weighted summation is performed on two images obtained through the x-space-based reconstruction, thereby converting a PSF with anisotropic resolution into a PSF with isotropic resolution.

The x-space-based reconstruction method performs the velocity normalization on the received voltage signal, followed by meshing the normalized voltage signal onto the scanning trajectory of the FFP. Compared with the system matrix-based reconstruction method, the x-space-based reconstruction method is characterized by low memory and fast scanning. However, because a device scanning trajectory of the x-space-based reconstruction method is a unidirectional Cartesian, and a magnetic field in a normal direction changes slowly due to excitation of a driving field, resolution of the PSF in the normal direction is lower than that in a collinear direction. As a result, spatial resolution of an MPI image is anisotropic, causing image quality degradation such as artifacts, signal-to-noise ratio reduction, and image shape distortion to the image obtained through the x-space-based reconstruction.

### SUMMARY

In view of this, embodiments of the present disclosure provide an MPI reconstruction method based on a time-domain system matrix and x-space, to resolve problems of spatial resolution anisotropy and artifacts in an x-space-based MPI reconstruction method in the prior art.

An embodiment of the present disclosure provides an MPI reconstruction method based on a time-domain system matrix and x-space, including:
obtaining a voltage signal through MPI scanning based on a Cartesian trajectory;
obtaining an original image according to an x-space-based reconstruction method;
constructing, based on voltage signal as well as a velocity compensation step and a grading step in the x-space-based reconstruction method, a forward model for time-domain-based MPI and x-space-based reconstruction; and
taking the original image as an input of an inverse problem solver of the forward model, and obtaining an optimized particle distribution diagram through solving by using an algebraic iteration method.

Optionally, the constructing, based on voltage signal as well as a velocity compensation step and a grading step in the x-space-based reconstruction method, a forward model for time-domain-based MPI and x-space-based reconstruction includes:
recovering, by using a fundamental frequency recovery algorithm, an excitation frequency component filtered out in a receiving process of the voltage signal;
constructing an MPI simulation model that retains a fundamental frequency signal; and
performing time and space discretization on the MPI simulation model to obtain a discrete time-domain system matrix S.

Optionally, the constructing, based on voltage signal as well as a velocity compensation step and a grading step in the x-space-based reconstruction method, a forward model for time-domain-based MPI and x-space-based reconstruction further includes:
weighting a unit element in each row of an initialized unit matrix, where a weighted value is a reciprocal of a velocity of an FFP corresponding to each sampling time point;
deleting a matrix row corresponding to a voltage signal whose scanning trajectory is outside a field of view (FOV) to obtain a system matrix V for implementing velocity compensation and deletion steps;
interpolating a normalized voltage signal onto a meshed scanning trajectory by using a nearest neighbor interpolation method; and
finding data corresponding to a time point with a closest Euclidean distance as an interpolation point of a pixel, and constructing a meshed system matrix G.

Optionally, the constructing, based on voltage signal as well as a velocity compensation step and a grading step in the x-space-based reconstruction method, a forward model for time-domain-based MPI and x-space-based reconstruction further includes:
performing data type and accuracy unification on the system matrix S, the system matrix V, and the system matrix G; and
multiplying the system matrix S, the system matrix V, and the system matrix G in sequence to obtain a system matrix TD-SM of the forward model.

Optionally, the algebraic iteration method uses a Kaczmarz iteration algorithm.

Optionally, the taking the original image as an input of an inverse problem solver of the forward model, and obtaining an optimized particle distribution diagram through solving by using an algebraic iteration method includes:
splitting the system matrix TD-SM by row, and regarding each row as an n-dimensional hyperplane; and
starting from an initial point, calculating a vertical projection of an original concentration distribution of a magnetic nanoparticle on each hyperplane sequentially until a concentration of the magnetic nanoparticle converges to a common intersection point of all hyperplanes.

Optionally, the recovering, by using a fundamental frequency recovery algorithm, an excitation frequency component filtered out in a receiving process of the voltage signal includes:
collecting the voltage signal through partial field-of-view (pFOV) scanning to obtain a pFOV image; and
recovering DC image intensity of the pFOV image.

Optionally, the performing time and space discretization on the MPI simulation model to obtain a discrete time-domain system matrix S includes:
taking sampling time as a time interval of the time discretization;
taking a pixel size of an MPI device as a voxel size of the space discretization;
obtaining a magnetization formula for a particle with a unit concentration at each location; and
obtaining the time-domain system matrix S based on a vacuum permeability, the magnetization formula for the particle, and a difference between sampling time points.

Optionally, the obtaining a voltage signal through MPI scanning based on a Cartesian trajectory method includes:
obtaining the voltage signal based on a vacuum permeability, an included angle between a direction of a magnetic field and a direction of a receiving coil, a magnetization response of a magnetic nanoparticle with a unit concentration, sensitivity of the receiving coil, a particle concentration, a temperature, and a magnetic moment of a single magnetic nanoparticle.

The embodiments of the present disclosure have following beneficial effects:

The embodiments of the present disclosure provide an MPI reconstruction method based on a time-domain system matrix and x-space. Based on an original image obtained through x-space-based reconstruction, a forward model describing MPI and x-space-based reconstruction processes is established by constructing a system matrix in a time domain and converting an x-space-based reconstruction method into a matrix operation. An iterative reconstruction algorithm is used to eliminate an impact of a PSF on an image obtained through the x-space-based reconstruction, achieving MPI image reconstruction with isotropic resolution and no artifact.

### BRIEF DESCRIPTION OF THE DRAWINGS

The features and advantages of the present disclosure can be more clearly understood with reference to the accompanying drawings. The accompanying drawings are illustrative and should not be understood as any limitation on the present disclosure. In the accompanying drawings:

FIG. 1 is a flowchart of an MPI reconstruction method based on a time-domain system matrix and x-space according to an embodiment of the present disclosure; and

FIGS. 2A-E show comparison results of an MPI reconstruction method based on a time-domain system matrix and x-space according to an embodiment of the present disclosure, where FIG. 2A shows an original image, FIG. 2B shows an image obtained through x-space-based reconstruction, FIG. 2C shows an image reconstructed by using a deconvolution method, FIG. 2D shows an image obtained through multi-channel signal collection, and FIG. 2E shows a reconstructed image in this embodiment.

### DETAILED DESCRIPTION OF THE EMBODIMENTS

In order to make the objectives, technical solutions, and advantages of the embodiments of the present disclosure clearer, the technical solutions in the embodiments of the present disclosure will be clearly and completely described below in conjunction with the accompanying drawings in the embodiments of the present disclosure. Apparently, the described embodiments are some, rather than all of the embodiments of the present disclosure. All other embodiments obtained by those of ordinary skill in the art based on the embodiments of the present disclosure without creative efforts should fall within the protection scope of the present disclosure.

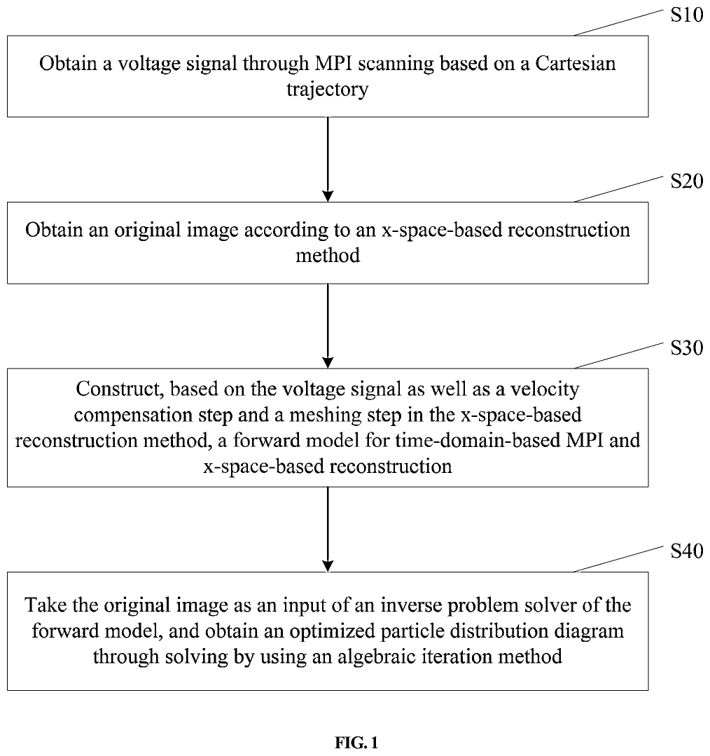

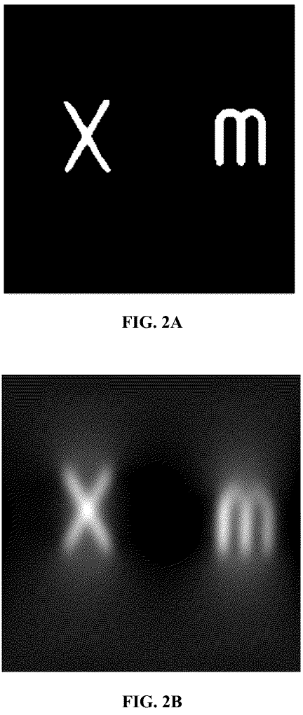

An embodiment of the present disclosure provides an MPI reconstruction method based on a time-domain system matrix and x-space Internet. As shown in FIG. 1, the MPI reconstruction method includes following steps.

Step S10: Obtain a voltage signal through MPI scanning based on a Cartesian trajectory.

In this embodiment, signal collection is performed on an induced voltage in a receiving coil of an MPI device.

In a specific embodiment, the voltage signal is obtained based on a vacuum permeability, an included angle between a direction of a magnetic field and a direction of the receiving coil, a magnetization response of a magnetic nanoparticle with a unit concentration, sensitivity of the receiving coil, a particle concentration, a temperature, and a magnetic moment of a single magnetic nanoparticle. The induced voltage in the receiving coil of the MPI device can be expressed as follows:

$$u ( t ) = - {μ}_{0} \cdot sin θ \cdot {\int }_{Ω} \frac{\partial M ( r , t )}{\partial t} \cdot p ( r ) \cdot c ( r ) dr$$

In the above formula, μ₀ represents the vacuum permeability, which is set to 4π×10⁻⁷ T·m/A; θ represents the included angle between the direction of the magnetic field and the direction of the receiving coil, which is set to

$$[ - \frac{π}{2} , \frac{π}{2} ] ;$$

M represents the magnetization response of the magnetic nanoparticle with the unit concentration; r represents a location; t represents time; p represents the sensitivity of the receiving coil, which is usually set to a constant value 1 for simplicity; and c represents a particle concentration at each different location, which usually ranges from 0 mg/ml to 5 mg/ml.

Under an adiabatic condition, the magnetization response M of the magnetic nanoparticle can be expressed by a Langevin formula:

$$\begin{matrix}M ( r , t ) = {N}_{p} m ℒ ( kH ( r , t ) ) \\ = {N}_{p} m ( coth ( kH ( r , t ) ) - \frac{1}{kH ( r , t )} )\end{matrix}$$

In the above formula,

$$k = \frac{{μ}_{0} m}{{k}_{B} T} ;$$

k_{B} represents a Boltzmann constant, which is set to 1.380649×10⁻²³ J/K; T represents the temperature, which ranges from 270 K to 310 K and is usually set to 300 K; [symbol] (⋅) represents a Langevin function; H represents strength of an applied magnetic field; m represents the magnetic moment of the single magnetic particle; and Nₚ represents a quantity of magnetic nanoparticles with the unit concentration at a specific location, which is usually set to 1-2×10¹⁶, depending on iron content.

Step S20: Obtain an original image according to an x-space-based reconstruction method.

In this embodiment, after the induced voltage signal is obtained, x-space-based reconstruction is adopted for the voltage signal. Standard x-space-based reconstruction is divided into two steps: a velocity compensation step and a grading step. In a first step, namely the velocity compensation step, the voltage signal is divided by a velocity of an FFP on a corresponding trajectory to normalize the voltage signal. In a second step, namely the grading step, a normalized voltage signal is interpolated onto a scanning trajectory of the FFP to obtain an image obtained through the x-space-based reconstruction.

Step S30: Construct, based on the voltage signal as well as the velocity compensation step and the grading step in the x-space-based reconstruction method, a forward model for time-domain-based MPI and the x-space-based reconstruction.

In this embodiment, although an excitation frequency component of the voltage signal is filtered out in a signal receiving process, the excitation frequency component can be recovered by using a fundamental frequency recovery algorithm. Based on this, an MPI simulation model that retains a fundamental frequency signal is established, making it feasible to construct a system matrix in a time domain.

For the velocity compensation step and the grading step in the x-space-based MPI image reconstruction method, the forward model is constructed by constructing the system matrix. The forward model describes a linear relationship between a particle concentration distribution in entire MPI and x-space-based reconstruction processes and the original image obtained through the x-space-based reconstruction. A non-negative property of a magnetic nanoparticle concentration can be obtained based on the forward model, namely a physical prior of the MPI.

Step S40: Take the original image as an input of an inverse problem solver of the forward model, and obtain an optimized particle distribution diagram through solving by using an algebraic iteration method.

In this embodiment, a non-negative constraint can be added in an iteration process, and a regularization method can also be added to improve accuracy and accelerate convergence.

Based on the original image obtained through the x-space-based reconstruction, by constructing the system matrix in the time domain and converting the x-space-based reconstruction method into a matrix operation, this embodiment establishes the forward model describing the scanning and the x-space-based reconstruction processes. An iterative reconstruction algorithm is used to eliminate an impact of a PSF on the image obtained through the x-space-based reconstruction, achieving MPI image reconstruction with isotropic resolution and no artifact.

In an optional implementation, the step S30 includes following substeps:

Step S301: Recover, by using the fundamental frequency recovery algorithm, the excitation frequency component filtered out in the receiving process of the voltage signal.

In this embodiment, the voltage signal is collected through pFOV scanning to obtain a pFOV image, and DC (direct current) image intensity of the pFOV image is recovered.

In a specific implementation, due to an impact of an excitation magnetic field in the received signal, a high-pass filter or a notch filter is required to filter out a fundamental-frequency voltage signal, which also results in a loss of fundamental frequency information in a particle response signal. An important assumption in the x-space-based reconstruction is that the MPI process is a linear shift invariance system. A loss of the fundamental frequency signal will make an MPI system no longer have this property. Therefore, a pFOV scanning method is adopted to divide an entire FOV divided into different patches. Then the voltage signal is collected. Finally, the fundamental frequency signal is recovered by recovering the DC image intensity of the pFOV image obtained through the x-space-based reconstruction.

Step S302: Construct the MPI simulation model that retains the fundamental frequency signal.

Step S303: Perform time and space discretization on the MPI simulation model to obtain a discrete time-domain system matrix S.

In this embodiment, sampling time is taken as a time interval of the time discretization; a pixel size of the MPI device is taken as a voxel size of the space discretization; a magnetization formula for a particle with the unit concentration at each location is obtained; and the time-domain system matrix S is obtained based on the vacuum permeability, the magnetization formula for the particle, and a difference between sampling time points. In a specific embodiment, the magnetization formula for of the particle with the unit concentration at each location is as follows:

$$M [ {r}_{i} , {t}_{j} ] = {N}_{p} m ( coth ( kH [ {r}_{i} , {t}_{j} ] ) - \frac{1}{kH [ {r}_{i} , {t}_{j} ]} )$$

In the above formula, i=1, 2, . . . , N, j=1, 2, . . . , Nₜ, N represents a quantity of discrete locations in the FOV, and Nₜ represents a quantity of sampling time points, which is equal to a result of scanning time divided by the sampling time.

The discrete time-domain system matrix S is expressed as follows:

$$S [ {r}_{i} , {t}_{j} ] = - {μ}_{0} \cdot \frac{M [ {r}_{i} , {t}_{j} ] - M [ {r}_{i} , {t}_{j - 1} ]}{Δ t}$$

As an optional implementation, the step S30 further includes following substeps:

Step S304: Weight a unit element in each row of an initialized unit matrix, where a weighted value is a reciprocal of a velocity of an FFP corresponding to each sampling time point.

Step S305: Delete a matrix row corresponding to a voltage signal whose scanning trajectory is outside the FOV to obtain a system matrix V for implementing velocity compensation and deletion steps.

Step S306: Interpolate the normalized voltage signal onto a meshed scanning trajectory by using a nearest neighbor interpolation method.

Step S307: Find data corresponding to a time point with a closest Euclidean distance as an interpolation point of a pixel, and construct a meshed system matrix G.

The standard x-space-based reconstruction is divided into the two steps: the velocity compensation step and the grading step. In the velocity compensation step, the voltage signal is divided by the velocity of the FFP on the corresponding trajectory to normalize the voltage signal. In the grading step, the normalized voltage signal is interpolated onto the scanning trajectory of the FFP to obtain the image obtained through the x-space-based reconstruction.

In order to convert the x-space-based reconstruction into a matrix operation, the system matrix is constructed in two steps in this embodiment. In the velocity compensation step, a signal at each time point is divided by a velocity of a corresponding FFP. The velocity v is determined by a form and a size of the magnetic field, in other words, the scanning trajectory. In fact, after the velocity compensation is performed on the voltage signal, a velocity of an FFP at a scanning edge is very small and close to 0 due to a sinusoidal shape of the scanning trajectory. After the velocity compensation is performed on the voltage signal, an artifact appears at the edge. Therefore, during scanning, a scanning trajectory of a driving field is usually set to be about 5% larger than that of the FOV. In this way, after the scanning, a voltage signal value outside the FOV is deleted, thus eliminating the artifact in the reconstructed image.

In a specific embodiment, an Nₜ×Nₜ unit matrix is initialized, and then a unit element in each row is weighted. A weighted value should be the reciprocal of the velocity of the FFP corresponding to each time point. After that, the matrix row corresponding to the voltage signal whose scanning trajectory is outside the FOV is deleted to obtain the system matrix V with the velocity compensation and deletion steps.

The normalized voltage signal is interpolated onto the meshed scanning trajectory by using the nearest neighbor interpolation method. After the applied magnetic field is determined, the scanning trajectory is also determined, and locations of different pixels in a final image are known. For each pixel, data corresponding to a time point with a closest Euclidean distance is found as an interpolation point to finally obtain the meshed matrix G.

As an optional implementation, the step S30 further includes following substeps:

Step S308: Perform data type and accuracy unification on the system matrix S, the system matrix V, and the system matrix G.

Step S309: Multiply the system matrix S, the system matrix V, and the system matrix G in sequence to obtain a system matrix TD-SM of the forward model.

In this embodiment, TD-SM=V·G·S. The system matrix TD-SM represents the linear relationship between the particle concentration distribution in the entire MPI and x-space-based reconstruction processes and the original image obtained through the x-space-based reconstruction.

As an optional implementation, the step S40 includes following substeps:

Step S401: Split the system matrix TD-SM by row, and regard each row as an n-dimensional hyperplane.

Step S402: Starting from an initial point, calculate a vertical projection of an original concentration distribution of the magnetic nanoparticle on each hyperplane sequentially until a concentration of the magnetic nanoparticle converges to a common intersection point of all hyperplanes.

In this embodiment, the algebraic iteration method uses a Kaczmarz iteration algorithm, which is expressed as follows:

$${x}^{k + 1} = {x}^{k} + \frac{{b}_{s} - {a}_{s}^{T} {x}^{k}}{{‖ {a}_{s} ‖}^{2}}$$

In the above formula, x represents an original concentration distribution of the magnetic particle; k represents a quantity of iterations; b represents the original image obtained through the x-space-based reconstruction; aₛ represents an s^{th} row of the system matrix TD-SM, where a value range of s is [1, N]; and N represents the quantity of discrete locations in the FOV.

In a specific implementation, the system matrix TD-SM is split by row, and each row is regarded as the n-dimensional hyperplane. Starting from the initial point, the vertical projection of the x on each hyperplane is calculated sequentially until the x converges to the common intersection point of all the hyperplanes.

If a spatial voxel size of the model is set to be consistent with a size of a pixel of the image obtained through the x-space-based reconstruction, the entire system matrix TD-SM will be a matrix with a same quantity of rows and columns. As a result, inverse problem solving will be a non-ill-posed problem, and the iteration algorithm of the inverse problem solving will converge quickly.

The non-negative property of the particle concentration can be obtained based on the physical prior of the MPI. Therefore, the non-negative constraint can be added in the iteration process, and the regularization method can also be added to improve the accuracy and increases a convergence speed.

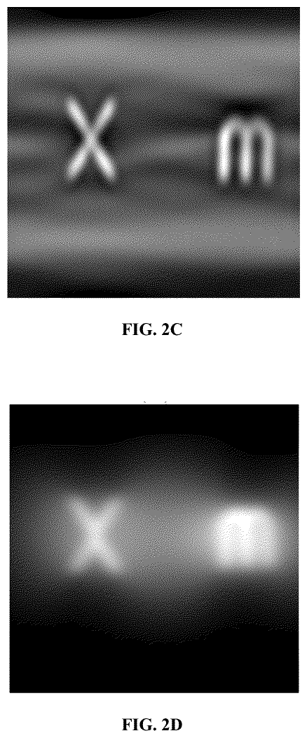

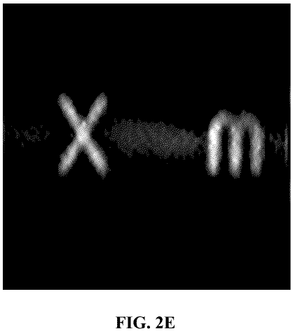

As shown in FIGS. 2A-E, FIG. 2A shows the original image, FIG. 2B shows the image obtained through the x-space-based reconstruction, FIG. 2C shows an image reconstructed by using a deconvolution method, FIG. 2D shows an image through multi-channel signal collection, and FIG. 2E shows the reconstructed image in this embodiment. The MPI reconstruction method based on a time-domain system matrix and x-space provided in the embodiments of the present disclosure effectively eliminates the impact of the PSF on the image obtained through the x-space-based reconstruction, greatly improves spatial resolution anisotropy of an imaging result under the Cartesian scanning trajectory, and does not introduce a new artifact while eliminating the artifact caused by the PSF, thereby achieving high-quality and high-resolution MPI image reconstruction.

Although the embodiments of the present disclosure are described with reference to the accompanying drawings, those skilled in the art may make various modifications and variations without departing from the spirit and scope of the present disclosure. These modifications and variations shall fall within the scope defined by the claims.

What is claimed is:

1. A magnetic particle imaging (MPI) reconstruction method based on a time-domain system matrix and x-space, comprising:
obtaining a voltage signal through MPI scanning based on a Cartesian trajectory;
obtaining an original image according to an x-space-based reconstruction method;
constructing, based on the voltage signal as well as a velocity compensation step and a grading step in the x-space-based reconstruction method, a forward model for time-domain-based MPI and x-space-based reconstruction; and
taking the original image as an input of an inverse problem solver of the forward model and obtaining an optimized particle distribution diagram through solving by using an algebraic iteration method.

2. The MPI reconstruction method based on a time-domain system matrix and x-space according to claim 1, wherein the constructing, based on the voltage signal as well as a velocity compensation step and a grading step in the x-space-based reconstruction method, a forward model for time-domain-based MPI and x-space reconstruction comprises:
recovering, by using a fundamental frequency recovery algorithm, an excitation frequency component filtered out in a receiving process of the voltage signal;
constructing an MPI simulation model that retains a fundamental frequency signal; and
performing time and space discretization on the MPI simulation model to obtain a discrete time-domain system matrix S.

3. The MPI reconstruction method based on a time-domain system matrix and x-space according to claim 2, wherein the constructing, based on the voltage signal as well as a velocity compensation step and a grading step in the x-space-based reconstruction method, a forward model for time-domain-based MPI and x-space reconstruction further comprises:
weighting a unit element in each row of an initialized unit matrix, wherein a weighted value is a reciprocal of a velocity of a field free point (FFP) corresponding to each sampling time point;
deleting a matrix row corresponding to a voltage signal whose scanning trajectory is outside a field of view (FOV) to obtain a system matrix V for implementing velocity compensation and deletion steps;
interpolating a normalized voltage signal onto a meshed scanning trajectory by using a nearest neighbor interpolation method; and
finding data corresponding to a time point with a closest Euclidean distance as an interpolation point of a pixel, and constructing a meshed system matrix G.

4. The MPI reconstruction method based on a time-domain system matrix and x-space according to claim 3, wherein the constructing, based on the voltage signal as well as a velocity compensation step and a grading step in the x-space-based reconstruction method, a forward model for time-domain-based MPI and x-space reconstruction further comprises:
performing data type and accuracy unification on the system matrix S, the system matrix V, and the system matrix G; and
multiplying the system matrix S, the system matrix V, and the system matrix G in sequence to obtain a system matrix TD-SM of the forward model.

5. The MPI reconstruction method based on a time-domain system matrix and x-space according to claim 4, wherein the algebraic iteration method uses a Kaczmarz iteration algorithm.

6. The MPI reconstruction method based on a time-domain system matrix and x-space according to claim 5, wherein the taking the original image as an input of an inverse problem solver of the forward model, and obtaining an optimized particle distribution diagram through solving by using an algebraic iteration method comprises:
splitting the system matrix TD-SM by row, and regarding each row as an n-dimensional hyperplane; and
starting from an initial point, calculating a vertical projection of an original concentration distribution of a magnetic nanoparticle on each hyperplane sequentially until a concentration of the magnetic nanoparticle converges to a common intersection point of all hyperplanes.

7. The MPI reconstruction method based on a time-domain system matrix and x-space according to claim 2, wherein the recovering, by using a fundamental frequency recovery algorithm, an excitation frequency component filtered out in a receiving process of the voltage signal comprises:
collecting the voltage signal through partial field-of-view (pFOV) scanning to obtain a pFOV image; and
recovering DC image intensity of the pFOV image.

8. The MPI reconstruction method based on a time-domain system matrix and x-space according to claim 2, wherein the performing time and space discretization on the MPI simulation model to obtain a discrete time-domain system matrix S comprises:
taking sampling time as a time interval of the time discretization;
taking a pixel size of an MPI device as a voxel size of the space discretization;
obtaining a magnetization formula for a particle with a unit concentration at each location; and
obtaining the time-domain system matrix S based on a vacuum permeability, the magnetization formula for the particle, and a difference between sampling time points.

9. The MPI reconstruction method based on a time-domain system matrix and x-space according to claim 1, wherein the obtaining a voltage signal through MPI scanning based on a Cartesian trajectory comprises:
obtaining the voltage signal based on a vacuum permeability, an included angle between a direction of a magnetic field and a direction of a receiving coil, a magnetization response of a magnetic nanoparticle with a unit concentration, sensitivity of the receiving coil, a particle concentration, a temperature, and a magnetic moment of a single magnetic nanoparticle.
